(19) United States
(12) Patent Application Publication
(10) Pub. No.: US 2017/0121697 A1
(43) Pub. Date: May 4, 2017

(54) METHOD FOR MAUFACTURING A STABLE AQUEOUS SOLUTION OF BETA-AMYLASE, AQUEOUS SOLUTION OBTAINED AND USES THEREOF

(71) Applicant: ROQUETTE FRERES, Lestrem (FR)

(72) Inventors: Vincent COURBOIS, Bethune (FR); Aline LECOCQ, Bethune (FR); Pierrick DUFLOT, La Couture (FR)

(73) Assignee: ROQUETTE FRERES, Lestrem (FR)

(21) Appl. No.: 15/319,079

(22) PCT Filed: Jun. 15, 2015
(86) PCT No.: PCT/FR2015/051581

§ 371 Date: Dec. 15, 2016

(30) Foreign Application Priority Data
Jun. 16, 2014 (FR) 14 55466

(51) Int. Cl. C12N 9/26; A23L 29/30; A23L 27/30; C12N 9/96

(52) U.S. Cl. CPC C12N 9/2425; C12Y 302/01002; C12N 9/96; A23L 29/35; A23L 27/30; A23V 2002/00

(57) ABSTRACT

The present invention relates to a method for stabilising an aqueous solution of β-amylase, in particular by the use of glycerol, potassium sorbate and sodium carbonate. The cocktail of additives is particularly effective at maintaining the enzymatic activity of β-amylase over time. Another aim of the present invention consists of using the cocktail for the specific function of maintaining the enzymatic activity of the β-amylase. Another aim of the present invention is to provide an aqueous solution of β-amylase containing the aforementioned cocktail. A final aim of the present invention consists of using the β-amylase aqueous solution in bread-making, in malting, as a food additive, as a digestive agent, for sweetener production, in pharmacy and, finally, for maltose and maltose-enriched syrup production.

The present invention relates to a method for manufacturing an aqueous solution of β-amylase, especially by using glycerol, potassium sorbate and sodium carbonate. This cocktail of additives proves to have particularly high performance with a view to maintaining the enzymatic activity of β-amylase over time.

Another subject of the present invention consists in the use of said cocktail with the specific function of maintaining the enzymatic activity of β-amylase. Another subject of the present invention consists of an aqueous solution of β-amylase containing the abovementioned cocktail. A final subject of the present invention consists in the use of said aqueous solution of β-amylase in bread making, in the malt industry, as food additive, as digestive agent, for the production of sweeteners, in pharmacy and finally for the production of maltose and maltose-enriched syrups.

β-amylases are exohydrolases which liberate maltose units from the non-reducing β ends of a 1→4 linked glucose polymers or oligomers, the reaction stopping at the first a 1→6 branching point encountered. Predominant components of “diastatic power” (corresponding to the combined activities of α-amylases, β-amylases, α-glucosidases and debranching enzymes) during malting (artificial germination of cereal grains), the β-amylase activities isolated from this enzymatic cocktail are essential for the production of maltose or other fermentable sugars generated from starch.

The saccharifying activity of just β-amylases is therefore exploited in a great number of applications: in bread making, in the malt industry, as food additive, or even as digestive agent, for the production of sweeteners, in pharmacy for the production of vaccines and finally for the production of maltose and maltose-enriched syrups (precursor of maltitol and maltitol syrups).

There are numerous methods for manufacturing β-amylases. Thus, it is known that ungerminated grains of barley, rye or wheat are all biological materials of choice for the large-scale commercial preparation of β-amylases. Moreover, it is known to those skilled in the art that half the (3-amylases which can be extracted from ungerminated grains of barley, wheat or rye may be readily obtained in the form of free enzymes by extraction with water and saline solutions. The other half is partially in the “bound” form which requires the addition of reducing agents or proteolytic enzymes for its extraction. Another fraction of β-amylases which cannot be directly extracted, referred to as “latent”, has also been described: detergents are required to extract it from cereal grains. Moreover, the methods for extraction of β-amylase described in the prior art are adapted depending on the targeted application.

In this respect, the applicant has developed, and protected in the application EP 2 414 379, an original method for producing β-amylases, in the sense that it relies on a hitherto barely exploited starting raw material: the “soluble fractions”. The latter were previously used exclusively as a source of nitrogen in fermentation and as a nutritive feed for livestock once said fractions had been enriched in fibers.

Such soluble fractions are produced during the wet extraction of the components of starch plants, such as corn, potato, sweet potato, wheat, rice, pea, broad bean, horse bean, cassava, sorghum, konjac, rye, buckwheat and barley. The components referred to as “noble”, produced during the extraction, are especially starches, proteins or else fibers. The “soluble fractions”, on the other hand, denote “non-noble” constituents: these are liquid residues resulting from said extraction, even though such residues may still contain, in trace amounts, rare insoluble substances and diverse and varied particles and colloids.

The method which is the subject of application EP 2 414 379 is based on the initial selection of the soluble fraction to be treated, then on a step of clarification carried out by microfiltration, and finally on a step of purification by ultrafiltration. On this occasion, it was demonstrated that the β-amylase obtained was particularly well suited to the preparation of maltose syrups, just like a β-amylase produced according to techniques according to the prior art but using more complex and costlier methods.

Following this, the applicant also protected improvements to this method through patent application FR 2 994 440 and French patent application no. 13 56022, as yet unpublished on the filing date of the present application. These improvements are based especially on the use of proteases during the microfiltration step, which makes it possible to very greatly reduce clogging of the microfiltration membranes and hence to increase the production time before washing, and also on the use of pectinases during the ultrafiltration step, which makes it possible to limit clogging of the ultrafiltration membranes, to reduce the viscosity of the retentate at the end of this step while increasing the richness thereof in β-amylase.

Now, the ultrafiltration retentate which contains the (3-amylase in concentrated form is able to be conserved for several weeks, or even several months, before being used in the applications which have already been mentioned. It then appears that its enzymatic activity reduces over time. It will be recalled that the activity of an enzyme is by definition the amount of substrate converted (or product formed) per unit time and under optimal operating conditions for the enzyme (temperature, pH, etc.). This value therefore quantifies the effectiveness of the enzyme.

Conventionally, enzyme activity is measured by determining another parameter, the diastatic activity. The latter is expressed in degrees diastatic power (° DP), defined as the amount of enzyme contained in 0.1 ml of a 5% by weight solution of a sample enzyme preparation sufficient for reducing 5 ml of Fehling's solution, when said sample is placed in 100 ml of substrate for 1 h at 20° C.

A number of documents are currently known describing methods for obtaining β-amylase with a view to improving the stability thereof from the perspective of the enzymatic activity thereof.

Document CN102965358 discloses a method for obtaining, from soy, a β-amylase by precipitation, then draining, clarification and ultrafiltration. Said method uses calcium chloride, and optionally salts of sulfuric acid, in the precipitation step.

Document CN102399763 describes the production of β-amylase from bran, with addition of calcium chloride and sodium hydrogen phosphate then concentration and stabilization in the presence of sorbitol and potassium sorbate, before sterilization.

Document CN101544967 discloses a method for manufacturing β-amylases by precipitation, separation and centrifugation, and then recommends adding calcium chloride, orthophosphoric acid, diatomaceous earths and glycerol.

Document CN1225943 describes a method for preparing β-amylase comprising the steps of ultrafiltration, concentration and precipitation of extracts of soy powder, the precipitation being preceded by the addition of sodium sulfate and the regulation of the pH between 3.6 and 5.

Document U.S. Pat. No. 2,496,261 describes a method for obtaining β-amylase from sweet potato, which comprises a step of precipitation in the presence of ammonium sulfate, then acidification with hydrochloric acid.

Document U.S. Pat. No. 4,024,000 describes a method for preparing β-amylase which uses divalent or trivalent ions chosen from calcium, magnesium, barium and aluminum hydroxides and salts thereof, and regulation of the pH within a range of between 4.5 and 8.

However, it should be noted that none of these solutions makes it possible to obtain a preparation of β-amylase in the form of an aqueous solution which is sufficiently stable over time, especially over periods of several weeks and more particularly for at least 70 days, from the perspective of its enzymatic activity. In the pursuit of its research, the applicant has succeeded in demonstrating that only a very particular selection of additives made it possible to achieve such a goal. This cocktail of additives consists of potassium sorbate, glycerol and sodium carbonate.

Thus, a first subject of the invention consists of a method for stabilizing an aqueous solution of β-amylase obtained from a soluble fraction of starch plants, comprising at least one step of introducing, into said aqueous solution of β-amylase:
a) potassium sorbate;
b) glycerol; and
c) sodium carbonate.

Advantageously, the potassium sorbate, the glycerol and the sodium carbonate are introduced into said aqueous solution of β-amylase in the following proportions:
a) from 0.05 to 0.5%, preferentially from 0.1 to 0.3%, and very preferentially approximately 0.2% potassium sorbate;
b) from 30 to 50%, preferentially from 35 to 45%, and very preferentially approximately 40% glycerol;
c) from 0.05 to 0.5%, preferentially from 0.1 to 0.3%, and very preferentially approximately 0.2% sodium carbonate;
   these % being expressed as % by dry weight of each constituent relative to the total weight of said aqueous solution.

Advantageously, the aqueous solution of β-amylase has a content by dry weight of β-amylase of between 5 and 20%, preferentially between 10 and 20%, very preferentially equal to approximately 15% relative to the total weight of said aqueous solution.

The potassium sorbate, the glycerol and the sodium carbonate are preferentially in the form of aqueous solutions. Those skilled in the art will know how to adapt the solids extract of these solutions, relative to the solubility of the products but also so as to limit the viscosity of these solutions and so as to make it easy to handle and especially pumpable.

According to one embodiment, the aqueous solution of β-amylase is obtained by the steps consisting in:
providing a soluble fraction of starch plants;
carrying out a microfiltration step on said soluble fraction, in order to obtain a microfiltration permeate;
carrying out an ultrafiltration step on the microfiltration permeate, in order to obtain an ultrafiltration retentate.

In this embodiment, it is therefore said ultrafiltration retentate which constitutes said aqueous solution of β-amylase, into which are introduced the potassium sorbate, the glycerol and the sodium carbonate.

In a more detailed way, and upstream of the method in accordance with the invention, it is suitable to choose the soluble fraction of starch plants to be treated. This selection is especially carried out from the group consisting of soluble fractions of corn, potato, sweet potato, wheat, rice, pea, broad bean, horse bean, cassava, sorghum, konjac, rye, buckwheat and barley.

The microfiltration of the soluble fraction of starch plant especially has the aim of eliminating insoluble substances, colloids, and microbiological material with a view to obtaining a clear composition containing β-amylase. The latter composition is therefore the microfiltration permeate. According to a particularly advantageous variant of the present embodiment, said microfiltration step is carried out in the presence of at least one protease. Prior to the microfiltration, the protease is placed in contact with the soluble fraction of starch plant to be treated: those skilled in the art will know how to adapt the contact time necessary for the action of the enzyme.

The protease used in the present invention is preferably chosen from serine proteases, thiol proteases, aspartyl proteases, and metalloproteases, and is more particularly chosen from the metalloproteases. By name, the proteases preferred in the present invention are the products sold under the names: Sumizyme™ APL, Lypaine™ 6500 L, Neutrase™ 0.8 L, Brewlyve™ NP 900, Brewers Clarex™. Preference will be given to using an amount of protease of between 0.01% and 0.1% by volume relative to the volume of the soluble fraction of starch plant to be treated.

The microfiltration step of the present embodiment is preferentially carried out by tangential membrane microfiltration. The applicant company more particularly recommends carrying out the tangential microfiltration with ceramic membranes having a porosity of 0.1 μm to 1 μm.

The microfiltration step may optionally be preceded by a step of flocculation of the insoluble particles contained in the soluble fraction of starch plants by any technique moreover known to those skilled in the art.

For this first microfiltration step, the applicant recommends working at a pH of between 4 and 5 and a temperature of between 40° C. and 50° C.

The microfiltration step is in particular controlled by the rise in transmembrane pressure (TMP) over time, with a fixed permeate flow rate.

In the present embodiment, the microfiltration is followed by an ultrafiltration step, firstly aiming to concentrate the microfiltration permeate containing the β-amylase, while removing from it any possible residual contaminating salts, sugars and proteins. The ultrafiltration is thus carried out on the microfiltration permeate so as to obtain an ultrafiltration retentate containing β-amylase.

The applicant company more particularly recommends carrying out the ultrafiltration by means of membranes having a cut-off threshold of 10 000 Da to 50 000 Da, preferably a cut-off threshold of 30 000 Da. The soluble fractions may for example be ultrafiltered on a module fitted with polysulfone membranes with a cut-off threshold of 30 000 Da in cassettes on the laboratory scale and polysulfone spiral membranes with a cut-off threshold of 30 000 Da on the pilot scale. The enzyme therefore becomes concentrated over time in the retentate.

This ultrafiltration may be carried out in the presence of pectinase. In the present application, the term pectinase denotes enzymes capable of decomposing pectins which are polysaccharide polymers and which are one of the constituents of plant cell walls. They are composed of a main chain of 1-4 linked uronic acid. In this respect, they should not be grouped together with cellulases and hemicellulases, with which they are erroneously associated:

cellulases are enzymes which participate directly in reactions for the decomposition of cellulose (linear chains of D-glucose molecules), while hemicellulases hydrolyze hemicellulose (branched sugar polymers in general, such as glucose, xylose, etc.).

The pectinase is typically introduced into the microfiltration permeate before carrying out the ultrafiltration step, and it is left to act.

Those skilled in the art will know how to adapt the contact time necessary for the action of the enzyme. The pectinase is typically left to act for from 30 minutes to 4 hours, preferentially from 30 minutes to 2 hours, at a temperature of between 25° C. and 60° C., preferentially between 25° C. and 50° C.

Pectinases which are particularly suited to the use of the present invention are the products Rapidase™ ADEX D (pectinase; DSM), Peclyve™ ESP (pectinase; Lyven), or Sumizyme™ ARS (pectinase and arabinase; Takabio), without these examples being in any way limiting.

It will be preferable to introduce an amount of pectinase of between 0.05% and 1% by volume relative to the total volume of the microfiltration permeate.

The ultrafiltration step may be followed by a step of dialysis of the ultrafiltration retentate so as to reduce the concentration of impurities in said retentate.

Moreover, it is desirable to maintain said solution of (3-amylase as obtained at a temperature of less than 15° C., preferentially of less than 10° C., ideally at approximately 5° C., so as to further improve the maintenance of its enzymatic activity.

Another subject of the present invention is the use, with a view to maintaining the enzymatic activity of β-amylase in an aqueous solution, of:
a) potassium sorbate;
b) glycerol; and
c) sodium carbonate.

“Maintaining the enzymatic activity” is intended to mean the ability to limit the drop in the degree ° DP as indicated in the experimental part. Typically, reference will be made to “maintaining” if the degree ° DP is still greater than at least 70% of its initial value, after 70 days at a temperature of 37° C.

Another subject of the present invention consists of a stabilized aqueous solution of β-amylase, containing:
a) potassium sorbate;
b) glycerol; and
c) sodium carbonate.

More particularly, this stabilized aqueous solution of β-amylase contains:
a) from 0.05 to 0.5%, preferentially from 0.1 to 0.3%, and very preferentially approximately 0.2% potassium sorbate;
b) from 30 to 50%, preferentially from 35 to 45%, and very preferentially approximately 40% glycerol;
c) from 0.05 to 0.5%, preferentially from 0.1 to 0.3%, and very preferentially approximately 0.2% sodium carbonate;
   these % being expressed as % by dry weight of each constituent relative to the total weight of said aqueous solution of β-amylase.

It also has a content by dry weight of β-amylase of between and 20%, preferentially between 10 and 20%, very preferentially approximately 15% relative to the total weight of said aqueous solution.

A final subject of the present invention consists of the use of the stabilized aqueous solution of β-amylase according to the invention in bread making, in the malt industry, as food additive, as digestive agent, for the production of sweeteners, in pharmacy for the production of vaccines, and finally for the production of maltose and of maltose-enriched syrups (precursor of maltitol and maltitol syrups).

The following examples make it possible to better understand the invention, without however limiting the scope thereof.

### EXAMPLES

### Manufacture of Aqueous Solutions of Beta-Amylase

Firstly, in the production of starch from wheat, a soluble fraction is taken off at the inlet to the solubles evaporator, which step is conventionally carried out to manufacture products intended for feeding livestock, once concentrated. These products are sold by the applicant company under the name Corami®. These soluble fractions have a pH of between 4 and 5 and a β-amylase activity of the order of 30° DP/ml.

Here, the soluble wheat fractions are microfiltered on pilot scale equipment. The microfiltration unit is fitted with ceramic membranes made of titanium oxide, the cut-off threshold of which is equal to 0.2 μm. The permeate flow rate is fixed at 12 l/(h m²). The volume concentration factor is equal to 1.5. The temperature and the pH of the permeate are equal to 45° C. and approximately 4.5, respectively.

0.8 l Neutrase (Novozymes) protease is added to the soluble fraction, at a fixed concentration of 0.1% by volume relative to the total volume of said composition. This protease is left to act beforehand for 1 hour at room temperature.

An ultrafiltration is then carried out as described above.

After 1 hour of microfiltration, a microfiltration permeate with a DP degree of 25° DP/ml is obtained, this degree reflecting the enzymatic activity of the solution containing the β-amylase. The enzymatic activity is measured by the diastatic activity. The latter is expressed in degree of diastatic power (° DP), defined as the amount of enzyme contained in 0.1 ml of a 5% by weight solution of a sample enzyme preparation sufficient for reducing 5 ml of Fehling's solution, when said sample is placed in 100 ml of substrate for 1 h at 20° C.

The microfiltration step is followed by an ultrafiltration step, carried out on the microfiltration permeate. The main aim of this step is to concentrate said permeate and to remove from it any possible residual contaminating salts, sugars and proteins. The ultrafiltration pilot equipment is fitted with organic polysulfone membranes with a cut-off threshold of 25 kDa (Alfa Laval membranes). The filtration temperature is fixed at 25° C. to limit bacterial growth as much as possible and preserve enzymatic activity. The transmembrane pressure (TMP) is fixed at 4 bar maximum.

An aqueous solution of β-amylase is thus obtained which consists of the ultrafiltration retentate, having a content by dry weight of β-amylase equal to 15% of the total weight thereof.

Different cocktails, as indicated in tables 1 to 3, were tested. All the % are expressed as % by dry weight of product relative to the total weight of the aqueous solution. Once the preparations have been produced, an enzymatic assay is carried out on each sample (contained in sterile 100 ml containers) according to the method described in patent application FR 2 994 440 (measure of the beta-amylase activity). This value serves as reference for the whole study. The different samples are then placed in a temperature-controlled oven at 37° C. for the desired period; a sample is then taken to measure the residual beta-amylase activity at different times (the days on which samples are taken are indicated in tables 1 to 3). The results are given in tables 1 to 3 and are expressed as % residual beta-amylase activity. The temperature of 37° C. is chosen so as to accelerate the phenomena which bring about the drop in enzymatic activity.

Table 1a demonstrates that the best result is obtained with the mixture of 40% glycerol, 0.2% potassium sorbate and 0.2% Na₂CO₃. It also demonstrates that compared to other cocktails using other ingredients, it is indeed the solution according to the invention which makes it possible to develop the best degree of stability. This is therefore indeed a non-obvious selection of ingredients to produce a cocktail which leads to surprising and entirely advantageous results in terms of limiting loss of enzymatic activity. Table 1a demonstrates that the cocktails as described in claim 1 of the present application make it possible to develop very high degrees of stability. The greatest stability is, moreover, obtained with the final cocktail described in this table, produced with the optimal doses of each ingredient, as described in claim 2 of the present application.

Table 2 demonstrates that glycerol, used alone and even at a high dose, does not make it possible to achieve the satisfactory degree of stability. Table 3 demonstrates that the substitution of glycerol with other sugars also does not make it possible to achieve a satisfactory degree of stability.

TABLE 1

| | | 50% | 40% | 40% | 40% | 50% |
|---|---|---|---|---|---|---|
| | 50% | glycerol + 0.2% | glycerol + 0.2% | sorbitol + 0.2% | sorbitol + 0.2% | glycerol + 0.2% |
| | glycerol + 0.2% | PS + 1% | PS + 1% | PS + 1% | PS + 1% | PS + 1% |
| Days | PS | Na₂HPO₄ | Na₂CO₃ | Na₂HPO₄ | CaCO₃ | CaCO₃ |
| 0 | 100 | 100 | 100 | 100 | 100 | 100 |
| 20 | 98 | 98 | | | | |
| 30 | | | 72 | | | |
| 34 | 89 | 89 | | 77 | 85 | 92 |
| 60 | | | 60 | | | |
| 72 | 48 | 70 | 75 | 66 | 69 | 70 |
| 90 | | 45 | 50 | 44 | 45 | 47 |

TABLE 1a

| | 60% | | 40% | 40% | 40% |
|---|---|---|---|---|---|
| | glycerol + | 40% | glycerol + | glycerol + | glycerol + |
| | 0.2% | glycerol + | 0.2% | 0.4% | 0.2% |
| | PS + | 1% PS + | PS + | PS + | PS + |
| | 0.4% | 0.4% | 0.4% | 0.2% | 0.2% |
| days | Na₂CO₃ | Na₂CO₃ | Na₂CO₃ | Na₂CO₃ | Na₂CO₃ |
| 0 | 100 | 100 | 100 | 100 | 100 |
| 72 | 74 | 73 | 76 | 76 | 80 |
| 90 | 49 | 48 | 54 | 54 | 60 |

TABLE 2*

| | 0% | 30% | 40% | 50% |
|---|---|---|---|---|
| | glycerol | glycerol | glycerol | glycerol |
| 0 | 100 | 100 | 100 | 100 |
| 30 | 0 | 53 | 65 | 69 |
| 60 | 0 | 27 | 44 | 56 |
| 90 | 0 | 7 | 16 | 28 |

TABLE 3

| | | 10% | 20% | 40% | 40% | | 40% mixture |
|---|---|---|---|---|---|---|---|
| | | glycerol + | glycerol + | glucose + | glucose + | | (45% glucose, |
| | 50% | 30% | 20% | 0.5% | 3% | 40% | 10% fructose, |
| Days | glucose | glucose | glucose | Na₂HPO₄ | NaCl | maltose | 45% maltose) |
| 0 | 100 | 100 | 100 | 100 | 100 | 100 | 100 |
| 30 | 66 | 53 | 61 | 82 | 42 | 46 | 39 |
| 60 | 43 | 35 | 37 | 43 | 15 | 30 | 20 |
| 90 | 28 | 22 | 22 | 22 | 7 | | |

PS: potassium sorbate

* the formation of a large insoluble deposit is also noted in the case of calcium carbonate

### Manufacture of Maltose Syrups

Two tests are then carried out, relating to the manufacture of maltose syrups from two aqueous solutions of beta-amylase stabilized by a cocktail according to the invention or by a cocktail not according to the invention, these 2 solutions having been kept for 90 days at 25° C. before being used.

A starch milk with 31% dry matter is liquefied in the conventional manner by means of 0.2% of an alpha-amylase (TERMAMYL120L sold by Novozymes) at a pH of 5.7 to 6.5 until a DE of roughly approximately equal to 6.

The reaction medium is then heated for a few seconds at 140° C. so as to inhibit the alpha-amylase, then the pH is adjusted to between 5 and 5.5 and the temperature to 55° C.

Saccharification is carried out at 35% dry matter, or slightly below, in the presence of pullulanase (PULLUZYME 750L sold by ABM) and maltogenic alpha-amylase (MALTOGENASE 4000L sold by Novozymes) and an aqueous solution of beta-amylase at doses equal to 0.1% of dry matter.

The aqueous solution of beta-amylase consists of the ultrafiltration retentate, having a content by dry weight of alpha-amylase equal to 15% of the total weight thereof, as described in the preceding example.

In a first test not according to the invention, this solution was stabilized with the cocktail according to the second column of table 1 (50% glycerol+0.2% PS+1% Na₂HPO₄). The solution remained at a temperature of 25° C. for 90 days before being used as indicated above.

In a second test according to the invention, this solution was stabilized with the cocktail according to the last column of table 1a (40% glycerol+0.2% PS+0.2% Na₂CO₃). The solution remained at a temperature of 25° C. for 90 days before being used as indicated above.

For these two tests, the saccharification, which lasts approximately 72 hours, gives a hydrolysate showing the following composition:

Not according to the invention:

### DP1: 2%, DP2: 77.9%, DP3: 5.6%

According to the invention:

### DP1: 5%, DP2: 88%, DP3: <1.5%

### Manufacture of Maltose Syrups

4 tests are then carried out, relating to the manufacture of maltose syrups from 4 stabilized aqueous solutions of beta-amylase. 3 tests according to the invention and one reference test were carried out using stabilized solutions having been stored for 90 days at 25° C. before being used.

A starch milk with 31% dry matter is liquefied in the conventional manner by means of 0.2% of an alpha-amylase (TERMAMYL120L sold by Novozymes) at a pH of 5.7 to 6.5 until a DE of approximately equal to 6.

The reaction medium is then heated for a few seconds at 140° C. so as to inhibit the alpha-amylase, then the pH is adjusted to between 5 and 5.5 and the temperature to 55° C.

Saccharification is carried out at 35% dry matter, or slightly below, in the presence of pullulanase (PULLUZYME 750L sold by ABM) and maltogenic alpha-amylase (MALTOGENASE 4000L sold by Novozymes) and an aqueous solution of beta-amylase at doses equal to 0.1% of dry matter.

The aqueous solution of beta-amylase consists of the ultrafiltration retentate, having a content by dry weight of beta-amylase equal to 15% of the total weight thereof, as described in the preceding example.

In a first test not according to the invention (CP), this solution was stabilized with the cocktail according to the second column of table 1 (50% glycerol+0.2% PS+1% Na₂HPO₄). The solution remained at a temperature of 25° C. for 90 days before being used as indicated above.

In a second test according to the invention (EX1), this solution was stabilized with the cocktail according to the last column of table 1a (40% glycerol+0.2% PS+0.2% Na₂CO₃). The solution remained at a temperature of 25° C. for 90 days before being used as indicated above.

In a third test according to the invention (EX2), this solution was stabilized with the cocktail according to the fourth column of table 1a (40% glycerol+0.4% PS+0.2% Na₂CO₃). The solution remained at a temperature of 25° C. for 90 days before being used as indicated above.

In a fourth test according to the invention (EX3), this solution was stabilized with the cocktail according to the third column of table 1 (40% glycerol+0.2% PS+1% Na₂CO₃). The solution remained at a temperature of 25° C. for 90 days before being used as indicated above.

For these tests, the saccharification, which lasts approximately 72 hours, gives a maltose syrup showing, for each of the examples, the following compositions:

Maltose syrup CP:

Glucose: 2%, maltose: 77.9%, maltotriose: 5.6%

Maltose syrup EX1:

Glucose: 5%, maltose: 88%, maltotriose: <1.5%

Maltose syrup EX2:

Glucose: 3.2%, maltose: 82.1%, maltotriose: 3.7%

Maltose syrup EX3:

Glucose: 2.8%, maltose: 81%, maltotriose: 4.6%

1. A method for stabilizing an aqueous solution of β-amylase obtained from a soluble fraction of starch plants, comprising at least one step of introducing, into said aqueous solution of β-amylase:
a) potassium sorbate;
b) glycerol;
c) sodium carbonate.

2. The method as claimed in claim 1, characterized in that the following are introduced into said aqueous solution of β-amylase:
a) from 0.05 to 0.5%, preferentially from 0.1 to 0.3%, and very preferentially approximately 0.2% potassium sorbate;
b) from 30 to 50%, preferentially from 35 to 45%, and very preferentially approximately 40% glycerol;
c) from 0.05 to 0.5%, preferentially from 0.1 to 0.3%, and very preferentially approximately 0.2% sodium carbonate;
these % being expressed as % by dry weight of each constituent relative to the total weight of said aqueous solution.

3. The method as claimed in claim 1, characterized in that the aqueous solution has a content by dry weight of β-amylase of between 5 and 20%, preferentially between 10 and 20%, very preferentially equal to approximately 15% of the total weight thereof.

4. The method as claimed in claim 1, characterized in that the potassium sorbate, the glycerol and the sodium carbonate are in the form of aqueous solutions.

5. The method as claimed in claim 4, wherein the aqueous solution of β-amylase is obtained by the steps consisting in:
providing a soluble fraction of starch plants;
carrying out a microfiltration step on said soluble fraction, in order to obtain a microfiltration permeate;
carrying out an ultrafiltration step on the microfiltration permeate, in order to obtain an ultrafiltration retentate.

6. (canceled)

7. An aqueous solution of β-amylase, containing:
a) potassium sorbate;
b) glycerol; and
c) sodium carbonate.

8. The aqueous solution as claimed in claim 7, containing:
a) from 0.05 to 0.5%, preferentially from 0.1 to 0.3%, and very preferentially approximately 0.2% potassium sorbate;
b) from 30 to 50%, preferentially from 35 to 45%, and very preferentially approximately 40% glycerol;
c) from 0.05 to 0.5%, preferentially from 0.1 to 0.3%, and very preferentially approximately 0.2% sodium carbonate;
these % being expressed as % by dry weight of each constituent relative to the total weight of said aqueous solution.

9. The aqueous solution as claimed in claim 7, having a content by dry weight of β-amylase of between 5 and 20%, preferentially between 10 and 20%, very preferentially approximately 15% of the total weight thereof.

10. A food additive or a digestive agent, comprising the aqueous solution as claimed in claim 7.

11. A method for the production of sweeteners, comprising adding the aqueous solution as claimed in claim 7 to a sweetener composition.

12. A method for the production of vaccines, comprising adding the aqueous solution as claimed in claim 7 to a vaccine.

13. A method for the production of maltose or maltose-enriched syrups, comprising adding the aqueous solution as claimed in claim 7 to maltose or a maltose-enriched syrup.
